(19) United States
(12) Patent Application Publication
(10) Pub. No.: US 2013/0011907 A1
(43) Pub. Date: Jan. 10, 2013

(54) Waste Management System

(75) Inventor: James Slanina, Chigago, IL (US)

(73) Assignee: 2245396 ONTARIO INC., Oakville (CA)

(21) Appl. No.: 13/534,020

(22) Filed: Jun. 27, 2012

Related U.S. Application Data
(60) Provisional application No. 61/504,772, filed on Jul. 6, 2011.

(51) Int. Cl. C12M 1/02; C12N 1/20

(52) U.S. Cl. 435/253.6; 435/290.2

(57) ABSTRACT

A waste management system is provided comprising a container having an internal chamber having a waste inlet adapted for receiving a supply of organic waste. A dry mix is adapted to be disposed within the internal chamber, the dry mix containing a dry substrate having a moisture content of less than 50% by weight. In another embodiment the dry mix contains a mixture of a dry substrate adapted for absorbing moisture from organic waste and a nutrient additive adapted for aiding in the decomposition of organic waste. The system further includes a drying system adapted for generating an airflow within the internal chamber and a mixing system adapted for mixing the contents of the internal chamber. The system also includes a control system adapted for controlling operation of the waste management system in accordance with a desired control process.

### FIELD

This specification relates to waste management systems and in particular to self contained systems for managing organic waste.

### BACKGROUND

The following background discussion is not an admission that anything discussed below is citable as prior art or common general knowledge. Any documents listed below are incorporated herein in their entirety by this reference to them.

Organic waste creates a wide range of environmental and food safety issues. Decomposing food in dumpsters, landfill and home composters emits undesired amounts of methane gas and attracts vermin and other disease spreading pests. Organic waste may be collected by waste management vehicles for processing at remote facilities but the use of such vehicles is inefficient and furthers the harm to the environment.

It is desirable that organic waste including food waste be managed onsite where the waste is produced, such as supermarkets, restaurants and other commercial or industrial food service locations as well as at home. Current onsite organic waste management systems do not manage the decomposition of the organic waste efficiently and/or may not be suitable for use in locations where water access and usage may be limited.

There is a need for an improved system that addresses these and other problems with prior existing systems.

### SUMMARY

In one aspect the invention provides a waste management system comprising:

a container having an internal chamber, said internal chamber having a waste inlet adapted for receiving a supply of organic waste;

a dry mix adapted to be disposed within said internal chamber, said dry mix containing a dry substrate having a moisture content of less than 50% by weight;

a drying system adapted for generating an air flow within said internal chamber;

a mixing system adapted for mixing the contents of said internal chamber; and

a discharge outlet adapted for facilitating periodic removal of the contents of said internal chamber.

In another aspect the invention provides a waste management system comprising:

a container having an internal chamber, said internal chamber having a waste inlet adapted for receiving a supply of organic waste;

a dry mix adapted to be disposed within said internal chamber, said dry mix containing a mixture of a dry substrate adapted for absorbing moisture from organic waste and a nutrient additive adapted for aiding in the decomposition of organic waste;

a drying system adapted for generating an air flow within said internal chamber;

a mixing system adapted for mixing the contents of said internal chamber; and

a discharge outlet adapted for facilitating periodic removal of the contents of said internal chamber.

In another aspect the invention provides a dry mix for use in a waste management system, said dry mix comprising:

a dry substrate adapted for absorbing moisture from organic waste, said dry substrate having a moisture content of less than 50% by weight; and

a nutrient additive adapted for aiding in the decomposition of organic waste.

Other aspects and features of the teachings disclosed herein will become apparent, to those ordinarily skilled in the art, upon review of the following description of the specific examples of the specification.

### DRAWINGS

The drawings included herewith are for illustrating various examples of articles, methods, and apparatuses of the present specification and are not intended to limit the scope of what is taught in any way. For simplicity and clarity of illustration, where considered appropriate, reference numerals may be repeated among the drawings to indicate corresponding or analogous elements.

FIG. 1 is a front view of a waste management system in accordance with an embodiment of the present invention;

FIG. 2 is a side view of the system of FIG. 1;

FIG. 3 is a side sectional view of the system as viewed along lines 3-3 of FIG. 1;

FIG. 4 is a front sectional view of the system as viewed along lines 4-4 of FIG. 2;

FIG. 5 is a front view of a waste management system in accordance with another embodiment of the present invention;

FIG. 6 is a side view of the system of FIG. 5;

FIG. 7 is a front sectional view of the system as viewed along lines 7-7 of FIG. 6; and

FIG. 8 is a process flow diagram depicting the process flow steps carried out by the control system for the systems of FIGS. 1 and 5.

### DESCRIPTION OF VARIOUS EMBODIMENTS

Various apparatuses or methods will be described below to provide examples of the claimed invention. The claimed invention is not limited to apparatuses or methods having all of the features of any one apparatus or method described below or to features common to multiple or all of the apparatuses described below. The claimed invention may reside in a combination or sub-combination of the apparatus elements or method steps described below. It is possible that an apparatus or method described below is not an example of the claimed invention. The applicant(s), inventor(s) and/or owner(s) reserve all rights in any invention disclosed in an apparatus or method described below that is not claimed in this document and do not abandon, disclaim or dedicate to the public any such invention by its disclosure in this document.

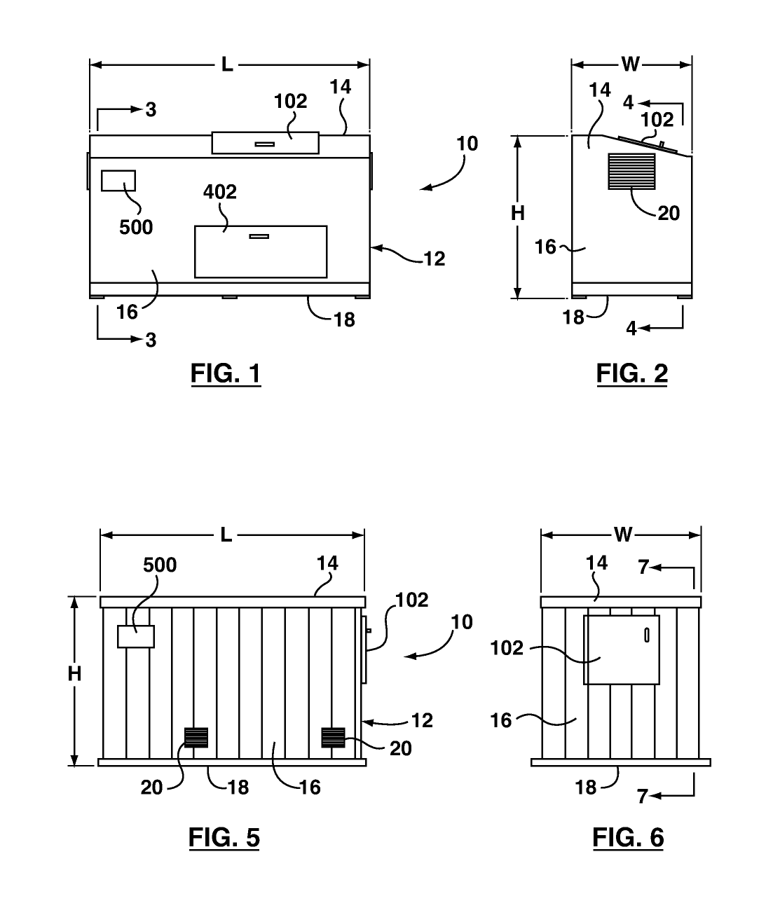

A waste management system in accordance with the present invention is shown generally at 10 in FIGS. 1 and 5. System 10 includes a container 12 having a top 14, sides 16, base 18 and one or more air vents 20.

Container 12 includes an internal chamber 22 defined by interior wall 24 for containing organic waste 26 and a dry mix 28 as described further below. Internal chamber preferably has a volume ranging from 0.33 cubic yards for home based systems to twelve cubic yards for commercial based units.

Dry mix 28 comprises approximately 20% to 50% of the volume of internal chamber 22, and more preferably between 25% and 40% of the volume and most preferably approximately 33% of the volume. Organic waste 26 comprises the same volume or a lesser volume of internal chamber 22 than dry mix 28 (while still ensuring that there is sufficient space within chamber 22 to allow for mixing of the contents and air flow). Most preferably, organic waste 26 comprises no more than approximately 33% of the volume of internal chamber 22, dry mix 28 comprises no more than 33% of the volume of internal chamber 22 and the remainder of chamber 22 is open space for allowing mixing and air flow.

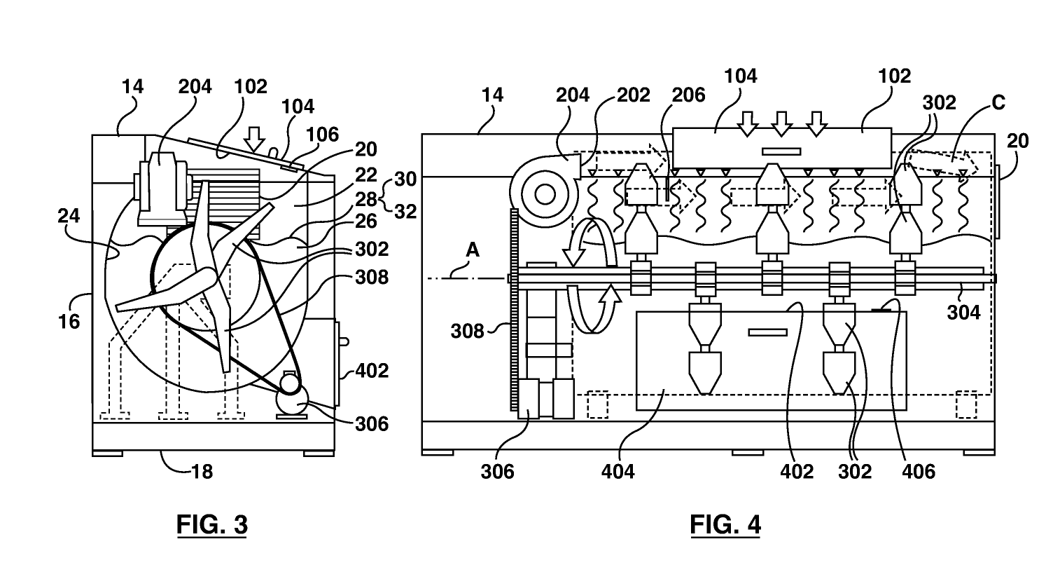

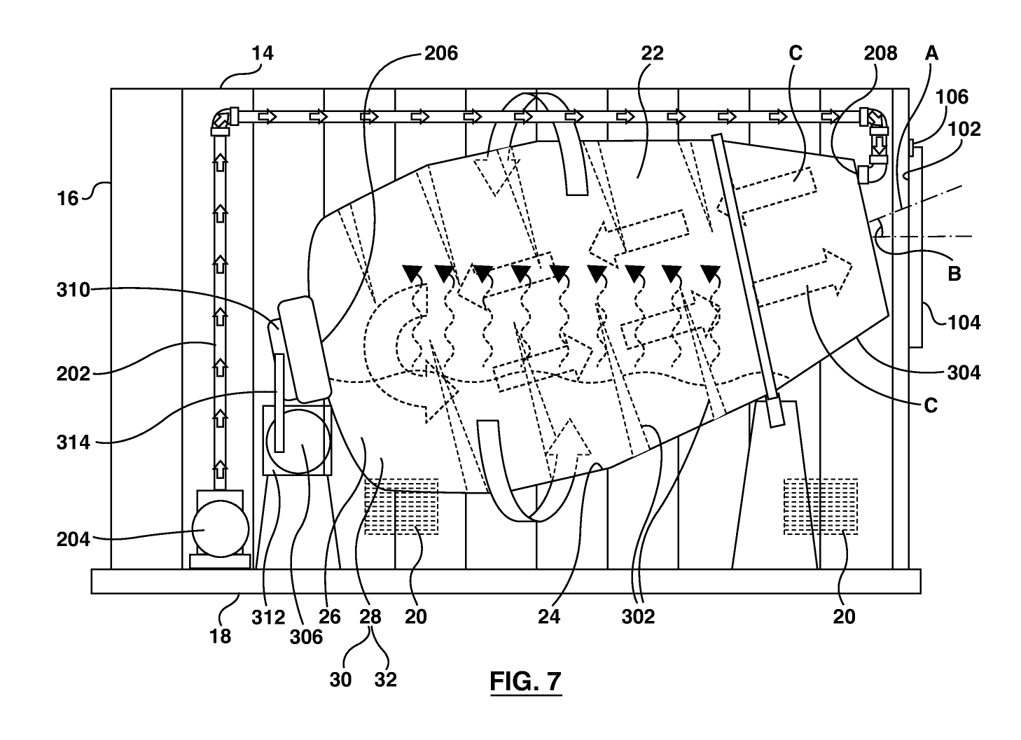

Interior wall 24 preferably has a rounded surface for encouraging mixing of organic waste 26 and dry mix 28 about a circulation axis A. For the system shown in FIGS. 1-4, circulation axis A preferably extends horizontally. For the system shown in FIGS. 5-7, circulation axis A extends at an incline angle B in the range of 20 to 40 degrees and most preferably in the range of 26 to 30 degrees.

Mid sized and large sized versions of system 10 are intended primarily for use at a supermarket, restaurant, hospital or other commercial or industrial food services facilities allowing organic waste 26 to be managed onsite. A scaled down version of the system is provided for home use.

Container 12 may be formed of metal (preferably stainless steel), plastic (such as high-density polyethylene) or other materials suitable for the intended use of system 10. Container is preferably weather proofed to prevent rain water from entering container 12 during use in outdoor locations.

Organic waste 26 may be comprised of yard waste such as leaves and plant or grass clippings and food waste such as leftover, expired, spoiled or other unwanted or unsafe food products, cooked or uncooked, typically derived from plant or animal sources including vegetables, fruits, meat, fish, poultry, dairy products, bones, shells, peels, pits, coffee grounds, fats, oils and greases.

Dry mix 28 is comprised of a dry substrate 30 combined with a nutrient additive 32 with a preferred ratio of approximately three parts dry substrate 30 for every one part nutrient additive 32 by weight (for example approximately thirty pounds of nutrient additive is mixed with approximately one hundred pounds of a wood blend dry substrate 30). Dry mix 28 has a relatively low moisture content that is preferably less than 50% by weight, more preferably less than 40% and most preferably less than 25%.

Dry substrate 30 comprises any suitable substrate material that provides sufficient surface area for absorbing water from organic waste 26 for subsequent evaporation. The substrate material also includes sufficiently sharp edges to assist in macerating organic waste 26 when it is mixed with dry mix 28 in internal chamber 22 to further increase the exposed surface area of the organic waste 26 to aid in evaporation and bacterial and biological decomposition.

More preferably dry substrate 30 comprises one or more organic materials such as wood chips, wood shavings, wood flour or sawdust (preferably cedar and preferably kiln dried). Most preferably dry substrate 30 comprises a blend of 20% cedar shavings, 20% 260 micrometer cedar sawdust (also known as wood flour), 20% 180 micrometer cedar sawdust, and 40% of ¾ inch chipped cedar all of which has been kiln dried to reduce moisture content to less than 25% by weight. This blend provides for proper absorption while providing maceration of organic waste 26 and maintaining sufficient separation of organic waste 26 to allow for proper airflow within chamber 22. Dry substrate 30 has a relatively low moisture content that is preferably less than 50% by weight, more preferably less than 40% and most preferably less than 25%.

Nutrient additive 32 comprises one or more nutrients that aid in the decomposition of organic waste 26. Nutrient additive 32 may contain nutrients such as Sulphate or TKN (Nitrogen), minerals (such as Copper, Magnesium, Potassium or Zinc), amino acids (such as Ascorbic, Benzoic or Lipoic) and vitamins (such as B-6/12, C, E, or K) that are preferably derived from natural plant sources and typically absent or lacking in sufficient quantities in organic waste 26. Nutrient additive 32 is preferably provided in a pellet or powder form and mixed with dry organic substrate 30 to form dry mix 28.

The nutrient additive 32 supplements nutrients that may already be contained within organic waste 26 and/or provides additional desired nutrients that aid in the decomposition of organic waste. It is believed that nutrient additive 32 provides at least one of the following advantages to the system 10: (a) promotes cellular division and proliferation of the bacterial populations responsible for decomposition (hydrolytic, acetifying, facultative bacteria); and/or (b) allows bacterial populations responsible for decomposition to out compete sulphide fixing bacteria (responsible for odours) for resources resulting in suppression of this bacterial population and/or (c) facilitates/catalyzes the hydrolytic rate limiting step in the biochemical reaction of the decomposition process in the bacterial populations.

System 10 further includes a waste inlet 100, a drying system 200, a mixing system 300, a discharge outlet 400 and a control system 500 all described further below.

Waste inlet 100 includes an access opening 102 defined in container 12 for providing access to deposit organic waste 26 into internal chamber 22. A door 104 is mounted to container 12 for closing access opening 102. Door sensor 106 is provided for sensing when door 104 is open or closed and communicating such information to control system 500.

Drying system 200 includes an outlet 202 that extends into internal chamber 22. Outlet 202 is connected to an air flow generator 204 that may be located inside or outside of container 12. Air flow generator 204 may be a blower or a vacuum for generating an air flow in the direction of arrows C within chamber 22. Vents 20 ensure a continuous supply of fresh air to chamber 22. Drying system 200 further includes a humidity sensor 206 disposed in internal chamber 22 and in communication with control system 500. Humidity sensor 206 senses when the humidity within internal chamber 22 drops below a predetermined level (preferably 40% relative humidity) at which point drying system 200 is no longer required for drying organic waste 26. For the embodiment of system depicted in FIGS. 5-7, drying system 200 further includes an oxygenation diffuser 208 disposed at the end of outlet 202.

Drying system 200 provides sufficient airflow within the internal chamber 22 to aid in reducing the moisture content of organic waste 26 through evaporation. Dry substrate 30 in the dry mix 28 further aids in reducing moisture content by absorbing moisture from the organic waste 22 and providing additional surface area for such moisture to evaporate. When organic waste 26 and dry mix 28 are mixed in chamber by mixing system 300 as described below the moisture content in organic waste 26 can be substantially reduced and the organic waste 26 broken down over time to a substantially dry nutrient rich compost material.

Mixing system 300 includes one or more contact elements 302, such as paddles, baffles or auger blades, for contacting, macerating and mixing organic waste 26 and dry mix 28 within inner chamber 22. Contact elements 302 are preferably mounted to a rotary element 304 such as a shaft (as provided for the embodiment in FIGS. 1-4) or a hollow drum (as provided for the embodiment of FIGS. 5-7). Rotary element 304 is connected to a motor 306 by means of a drive chain 308. A reducer gear 310 may also be provided to achieve a desired rate of circulation. A rate of circulation of around 3 RPMs is preferred. Motor 306 is preferably a 220 volt, three phase electric motor that ranges from ⅓ HP for smaller capacity systems (eg 0.33 cubic yards) to 25 HP for larger capacity systems (eg 12 cubic yards). For the embodiment of system depicted in FIGS. 5-7, mixing system 300 further includes a hydraulic pump 312 and a hydraulic motor 314 for assisting in moving rotary element 304.

Discharge outlet 400 includes an access opening 402 defined in container 12 for providing access to internal chamber 22 for periodic removal of the mixture of dry mix and dried organic waste. For the embodiment depicted in FIGS. 1-4, a lockable door 404 is mounted to container 12 for closing access opening 402. Door sensor 406 is provided for sensing when door 404 is open or closed and communicating such information to control system 500. For the embodiment depicted in FIGS. 5-7, waste inlet 100 and discharge outlet 400 have the same location and the mixture of dry mix and dried organic waste is periodically removed by reversing the direction of rotation for mixing system 300 in order that the auger blade contact elements 302 direct material out of internal chamber 22.

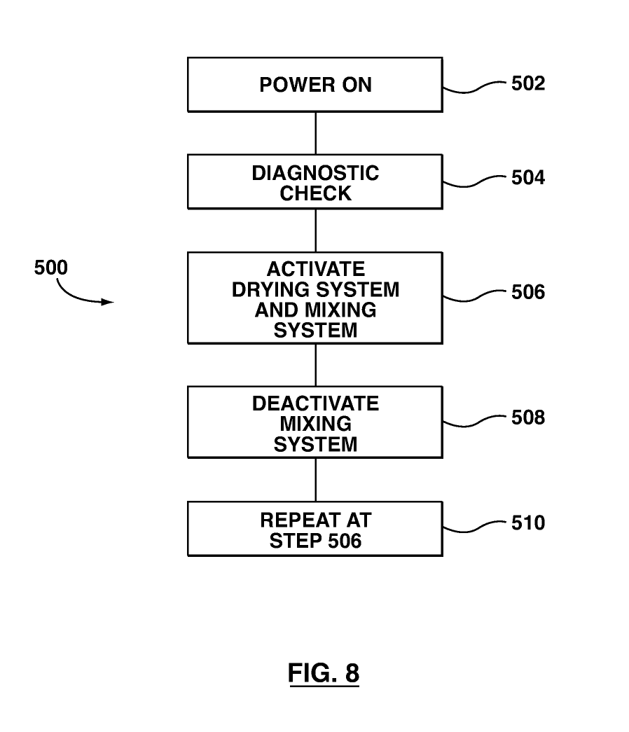

Control system 500 is provided for controlling operation of the organic waste disposal system 10 in accordance with a desired process of operation such as described below and shown in FIG. 8.

Process begins with step 502 where the system is powered on. The system then proceeds to step 504 and performs a diagnostic check with door sensors 106 and 406 to ensure that doors 104 and 404 are closed, with humidity sensor 206 to ensure that the relative humidity within internal chamber 22 is above a predetermined level and with drying system 200 and mixing system 300 to ensure that each are operational. If the diagnostic check identifies any error conditions (door open or non functioning air flow generator 204 or motor 306) the system goes into fault mode and will shut down further operation until the error condition is resolved. If the diagnostic check determines that the humidity level is below the predetermined amount it will shut down operation of the drying system 200 until the humidity level rises above the predetermined amount.

The system then proceeds to step 506 and activates drying system 200 and mixing system 300 for a preferred period of three minutes to mix the contents of internal chamber 22 and macerate organic waste 26. The control system then proceeds to step 508 and shuts down the mixing system 300 preferably for a period of fifteen minutes to allow time for evaporation of moisture from the exposed surfaces of the dry mix 28 and organic waste 26 in internal chamber 22. Drying system 200 continues to operate during step 508 to accelerate the rate of evaporation within internal chamber 22 unless the relative humidity within chamber 22 has dropped below the predetermined level.

The system then repeats process steps 506 and 508 while continually monitoring door sensors 106 and 406 and humidity sensor 206. If doors are opened the control system will shut down the drying system 200 and mixing system 300 until the doors are closed and if humidity level is below the predetermined amount the control system will shut down the drying system 200 until the humidity level rises above the predetermined level.

The system is adapted to operate continuously while additional organic waste 26 is added periodically through door 102. The mixture of dry mix 28 and dried organic waste 26 may be removed from the internal chamber 22 through discharge outlet 400 periodically (preferably every four to six months if used each day).

While the above description provides examples of one or more processes or apparatuses, it will be appreciated that other processes or apparatuses may be within the scope of the accompanying claims.

I/We claim:

1. A waste management system, comprising:
a container having an internal chamber, said internal chamber having a waste inlet adapted for receiving a supply of organic waste;
a dry mix adapted to be disposed within said internal chamber, said dry mix containing a dry substrate having a moisture content of less than 50% by weight;
a drying system adapted for generating an air flow within said internal chamber;
a mixing system adapted for mixing the contents of said internal chamber; and
a discharge outlet adapted for facilitating periodic removal of the contents of said internal chamber.

2. The system of claim 1, wherein said dry substrate comprises wood chips.

3. The system of claim 2, wherein said dry substrate further comprises one or more of wood shavings, wood flour or saw dust.

4. The system of claim 1, wherein sufficient dry mix is provided to fill approximately 33% of the volume of said internal chamber.

5. The system of claim 1, wherein said dry mix further comprises a nutrient additive adapted for aiding in the decomposition of organic waste.

6. A system as claimed in claim 5 wherein said nutrient additive contains one or more minerals, amino acids or vitamins selected to aid in the decomposition of said organic waste.

7. A system as claimed in claim 1, wherein said dry substrate has a moisture content of less than 40% by weight.

8. A system as claimed in claim 1, wherein mixing system comprises at least one contact element that is disposed on a rotary element for mixing the contents of said internal chamber.

9. A system as claimed in claim 8, wherein said rotary element comprises a shaft disposed within said internal chamber.

10. A system as claimed in claim 8, wherein said rotary element comprises a drum that defines said internal chamber, said at least one contact element being disposed on an interior wall of said drum.

11. A system as claimed in claim 1, further comprising a control system adapted for controlling operation of said waste management system in accordance with a desired control process.

12. A waste management system, comprising:
a container having an internal chamber, said internal chamber having a waste inlet adapted for receiving a supply of organic waste;
a dry mix adapted to be disposed within said internal chamber, said dry mix containing a mixture of a dry substrate adapted for absorbing moisture from organic waste and a nutrient additive adapted for aiding in the decomposition of organic waste;
a drying system adapted for generating an air flow within said internal chamber;
a mixing system adapted for mixing the contents of said internal chamber; and
a discharge outlet adapted for facilitating periodic removal of the contents of said internal chamber.

13. A system as claimed in claim 12, wherein said nutrient additive contains one or more minerals, amino acids or vitamins selected to aid in the decomposition of organic waste.

14. A system as claimed in claim 12, wherein said dry substrate has a moisture content of less than 50%.

15. A system as claimed in claim 12, wherein mixing system comprises at least one contact element that is disposed on a rotary element for mixing the contents of said internal chamber.

16. A system as claimed in claim 15, wherein said rotary element comprises a shaft disposed within said internal chamber.

17. A system as claimed in claim 15, wherein said rotary element comprises a drum that defines said internal chamber, said at least one contact element being disposed on an interior wall of said drum.

18. A dry mix for use in a waste management system, said dry mix comprising:
a dry substrate adapted for absorbing moisture from organic waste, said dry substrate having a moisture content of less than 50% by weight; and
a nutrient additive adapted for aiding in the decomposition of organic waste.

19. A dry mix as claimed in claim 18 wherein said dry substrate includes a mixture of wood chips and one or more of wood shavings, wood flour and sawdust.

20. A dry mix as claimed in claim 18 wherein said nutrient additive contains one or more minerals, amino acids or vitamins selected to aid in the decomposition of said organic waste.
